(19) United States
(12) Patent Application Publication
(10) Pub. No.: US 2024/0000946 A1
(43) Pub. Date: Jan. 4, 2024

(54) CARBONIC ANHYDRASE INHIBITORS SYNTHESIZED ON INTERCONNECTING LINKER CHAINS

(71) Applicant: VILNIUS UNIVERSITY, Vilnius (LT)

(72) Inventors: Daumantas MATULIS, Vilnius (LT); Audrius ZAKSAUSKAS, Vilnius (LT); Asta ZUBRIENE, Vilnius (LT); Lina BARANAUSKIENE, Vilnius (LT); Jurgita MATULIENE, Vilnius (LT); Virginija DUDUTIENE, Vilnius (LT); Edita CAPKAUSKAITE, Vilnius (LT); Vaida PAKETURYTE, Vilnius (LT); Joana SMIRNOVIENE, Vilnius (LT)

(21) Appl. No.: 18/039,644

(22) PCT Filed: Dec. 3, 2021
(86) PCT No.: PCT/IB2021/061310

§ 371 Date: May 31, 2023

Related U.S. Application Data
(60) Provisional application No. 63/120,734, filed on Dec. 3, 2020.

(51) Int. Cl. A61K 47/54; A61K 47/60; A61P 35/00

(52) U.S. Cl. CPC A61K 47/54; A61K 47/60; A61P 35/00

(57) ABSTRACT

This invention relates to a field of novel multi-headed compounds capable of binding and inhibiting the catalytic activity of human carbonic anhydrases for diagnostic, visualization, and treatment purposes. A series of compounds containing two or more of carbonic anhydrase inhibitors are linked together via short or long linker molecular moieties. Such dimeric or multimeric inhibitors are designed in such a way that one inhibitor molecule is able to reach several CA IX molecules on the surface of cancer cells. Such compounds are expected to have a significantly more powerful therapeutic effect and may be used for various strategies of specific compound deliveries to the desired site.

### FIELD OF THE INVENTION

This invention relates to a field of novel multi-headed compounds capable of binding and inhibiting the catalytic activity of human carbonic anhydrases for diagnostic, visualization, and treatment purposes.

### BACKGROUND OF THE INVENTION

Humans contain 15 isoforms of carbonic anhydrases that belong to the alpha family. Only twelve of these isoforms are catalytically active. Three are inactive because they lack Zn(II) in the active site due to mutations of His residues that hold the Zn(II). Human CA isoforms exhibit different cellular localization and multimerization patterns. Isoforms CA I, CA II, CA Ill, CA VII, and CA XIII are found intracellular, CA VA and CA VB are mitochondrial, CA VI is excreted, while the remaining ones are expressed at the outer surface of the cell membrane and attached via transmembrane domain (CA IX, CA XII, and CA XIV) or lipid linker (CA IV). The CA VIII, CA X, and CA XI are catalytically inactive. The genetics, molecular biology, enzymology, and industrial applications of CA are well described in numerous books¹⁻⁵.

Small molecular size chemical compounds that possess primary sulfonamide group inhibit human carbonic anhydrases by binding to the catalytic Zn(II) and thus preventing the binding of substrate CO₂. Primary sulfonamides are strong inhibitors of carbonic anhydrases and have become clinically used drugs to treat hypertension and edema, and be used as diuretics. Dorzolamide and brinzolamide have been used as antiglaucoma agents. The isoform CA IX together with several other isoforms have been shown to be highly overexpressed in numerous cancers. Thus they are thought to be targeted both for cancer visualization/diagnostics and possibly for treatment by inhibiting their catalytic activity.

There is a number of novel compounds that are supposed to inhibit CA IX described in the literature. However, it appears that all of them possess a weak affinity towards CA IX to efficiently inhibit the acidification of extracellular medium. We have previously designed a series of compounds that selectively and with high affinity interact with selected isoforms of CA, especially the isoform CA IX that is highly overexpressed in numerous cancers. The compounds possessed affinity in the range of 10-100 pM towards human CA IX in vitro^{6,7}.

These compounds have shown promise as potential anticancer agents and have been already applied for cancer diagnostic/visualization purposes⁸⁻¹⁰. However, their affinities are likely still way too weak for efficient prevention of extracellular acidification in cancer cell culture mediums.

### SUMMARY OF THE INVENTION

Here we designed a series of compounds where one, two, or more of the above-mentioned inhibitors are linked together via short or long linker molecular moieties. Such monomeric, dimeric or multimeric inhibitors are designed so that one inhibitor molecule can reach one or several CA IX molecules on the surface of cancer cells. FIG. 1 shows the principle of the design of such inhibitors.

An inhibitor consists of:
Head-groups
   Selective, specific, high-affinity compound able to recognize a particular CA isoform
   Reporter groups, such as IR fluorescent groups, for optically guided surgery
   Killer groups, such as PET, used for cancer treatment
   Anchor groups, such as biotin, for cell sorting and similar applications
Linker network
   Single linker connecting two head-groups
   Network of linkers connecting two or more identical or different head-groups.

Such an inhibitor is expected to have a significant advantage over a single-head group forming compound because two or more compounds will bind much more strongly to the target protein than the single compound. Such compounds are expected to have a significantly more powerful therapeutic effect and may be used for various strategies of specific compound deliveries to the desired site. For example, the compounds could be used for optically guided cancer surgery or for the delivery of PET groups that can be used for diagnostics or cancer cell destruction. The compounds may also be specific cell killer agents such as tubulysin and others.

However, most importantly, such novel double-headed compounds described in this invention will bind many orders of magnitude more strongly to CA IX on the cell surface than single-headed compounds because two heads will cooperatively enhance each other's effect. When one head dissociates, another one remains bound, and then the first will rebind much more rapidly than if being alone.

In one embodiment of the invention we present a conjugate of formula Q-L-FG, wherein Q is a binding ligand of carbonic anhydrase IX, L is an optional linker, and FG is a functional group, therapeutic agent or an imaging agent.

In another embodiment we present a conjugate of formula FG0-1-L-Q2-8, containing at least two Q, wherein Q is a binding ligand of carbonic anhydrase IX, L is an optional linker, and the linker is unsubstituted or substituted by one or more identical or different FG groups, which are selected from functional groups, therapeutical agents or an imaging agents.

### BRIEF DESCRIPTION OF THE DRAWINGS

FIG. 1. Schematic visualization of the main idea of this patent application. Top part shows that the compound would consist of one or more ‘heads’, previously discovered, specific, and high-affinity compounds, and a ‘linker’ or linker network connecting any number of heads. The bottom part shows an example of how such s networked compound could reach several CA IX protein molecules and simultaneously bind and inhibit them. The compound may consist of various other linked parts, such as a PET group (both for treatment and diagnostics), and a fluorescent group (both for diagnostics and visualization). The system may also be applied for optically guided cancer surgery.

FIG. 2. ¹H-NMR spectrum of AZ19-3-2 in DMSO-D6 solvent shows the observed peak's ppm shifts and their integration. The spectrum is part of the tests to identify and confirm the structure of the compound.

FIG. 3. Determination of affinity of compound AZ19-3-2 for recombinant human carbonic anhydrase catalytic domains using the fluorescent thermal shift assay. Graphs show the protein melting temperatures as a function of added compound concentration. An increase in Tₘ is proportional to the affinity constant. The lines were fit according to the thermodynamic model.

FIG. 4. Determination of affinity of compound AZ19-3-2 for recombinant human carbonic anhydrase catalytic domains using the fluorescent thermal shift assay.

FIG. 5. The graph shows the inhibition of the enzymatic activity of carbonic anhydrase IX by the double-headed AZ19-3-2 as a function of added compound concentration. The 50% remaining activity is observed at approximately 10 nM concentration which coincides with the position where double-headed compound is expected to inhibit half of the 50 nM enzyme CA IX concentration. This shows that the compound binds stronger than the 10 nM and titrates the enzyme at this concentration. Therefore, the affinity cannot be determined by this technique. It only shows that the real affinity is somewhere below 10 nM. Thus, we confirm by this technique that the compound is an inhibitor of CA IX enzymatic activity, but the affinity constant can be determined only by other methods such as the fluorescent thermal shift assay. The resultant affinities for all human CA isoforms are listed in Table 1.

FIG. 6. HeLa cancer cell culture grown for 48 hours under hypoxic (1% O₂) conditions caused acidification of extracellular medium as visualized by pH drop from initial pH 7.74 (shown as a dashed bold line) to pH 6.35. Compounds inhibited this acidification depending on the concentration of compounds. The headgroup compound VD11-4-2 (open circles connected by a dotted line as a guide for an eye) was only partially capable of inhibiting the acidification even at high 60 μM concentration (pH 7.3) while the double-headed AZ19-3-2 compound (gray filled triangles connected by solid line as a guide for an eye) inhibited the acidification effect fully at concentrations as low as approximately 10 μM.

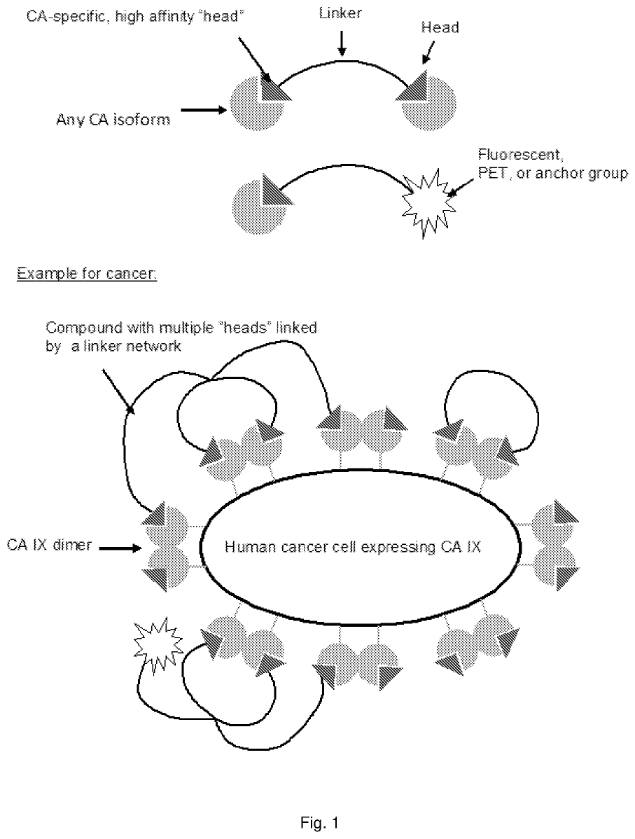

### DETAILED DESCRIPTION OF THE INVENTION

Organic Synthesis of Compounds

Below are several schemes showing the chemical structures of some suggested and synthesized compounds tested in vitro where their affinities have been determined for all human CA isoforms and tested in human cancer cell cultures.

Scheme 1. Possible head-groups (1-6) designed as compounds that specifically and with high affinity binding to human carbonic anhydrases, especially CA IX.

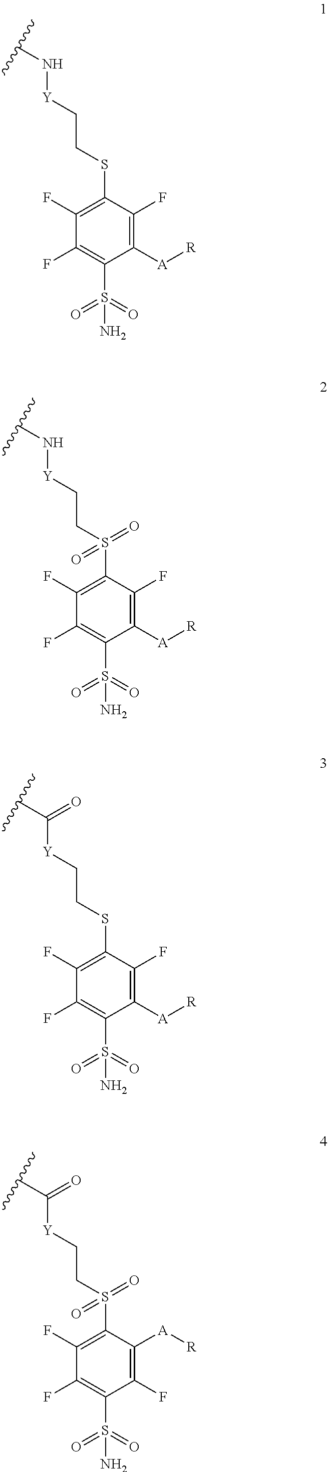

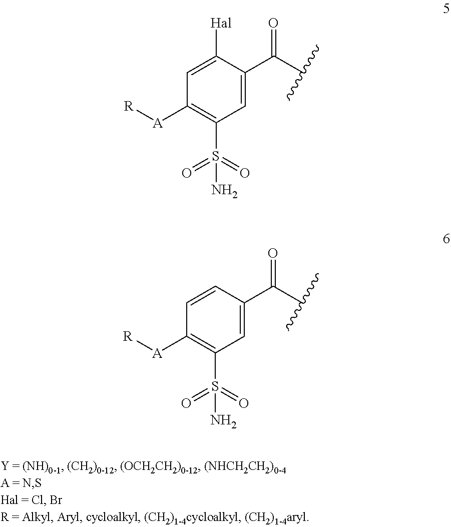

Scheme 2. This scheme shows possible chemical structures of the compounds bearing various head-groups attached by a linker such as PEG. The linker system may not be limited to two-ends but could be branched with many branches depending on the purpose of the compound.

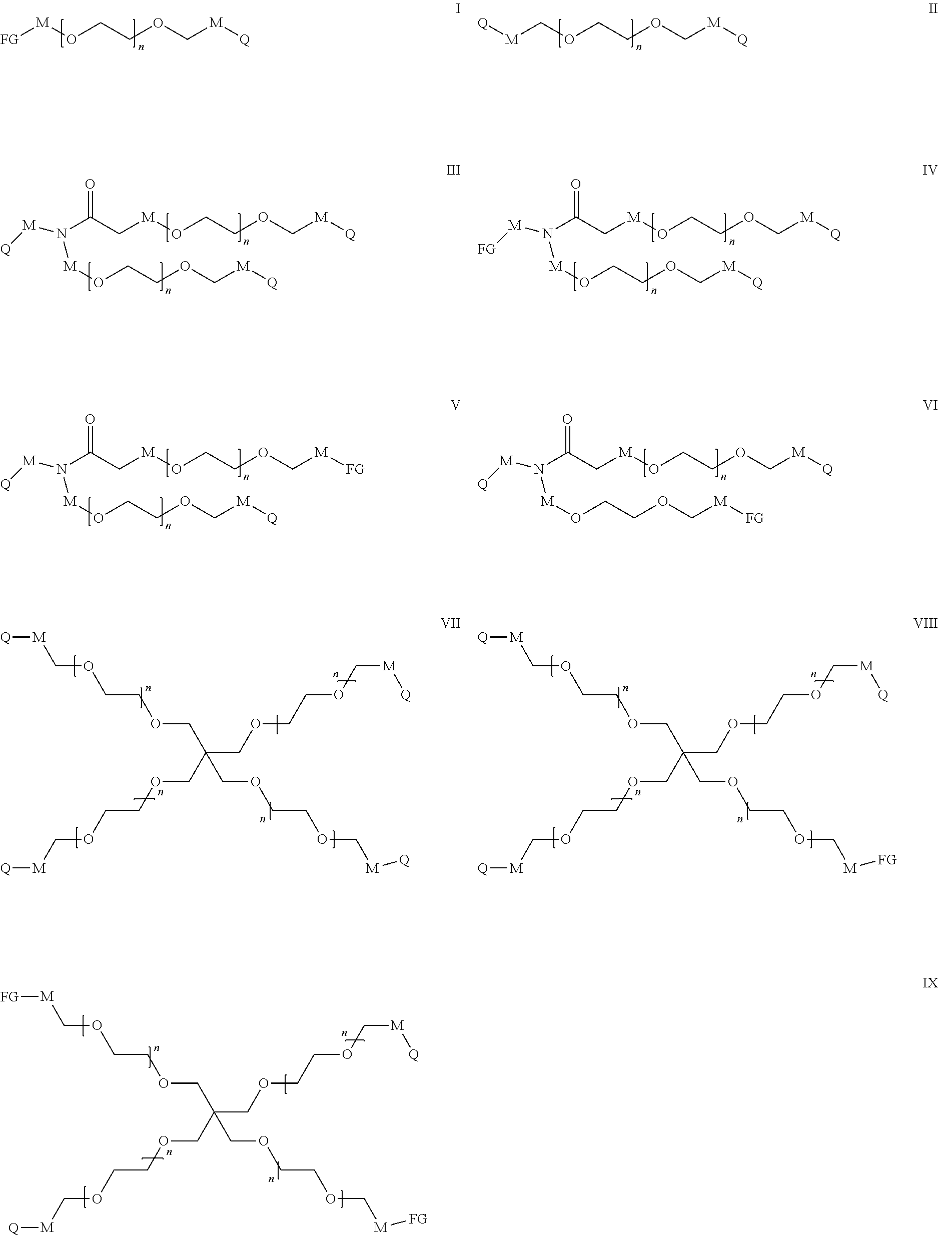

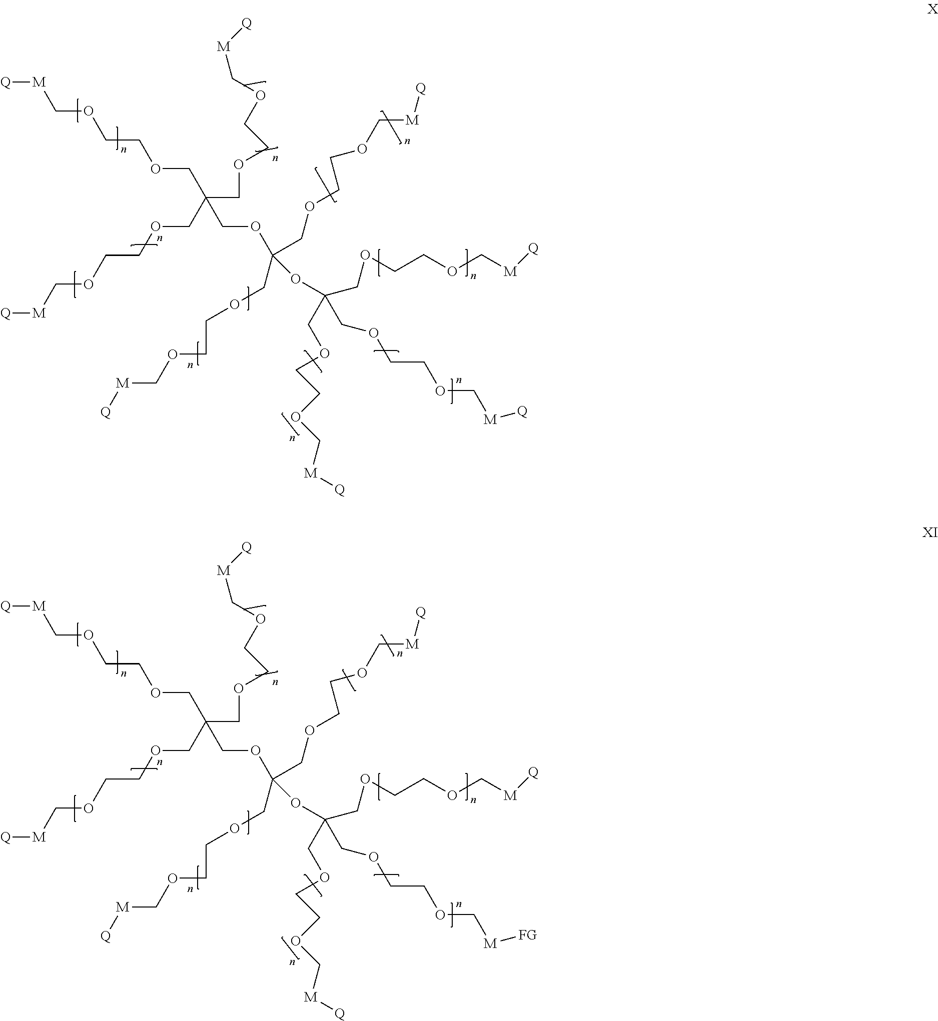

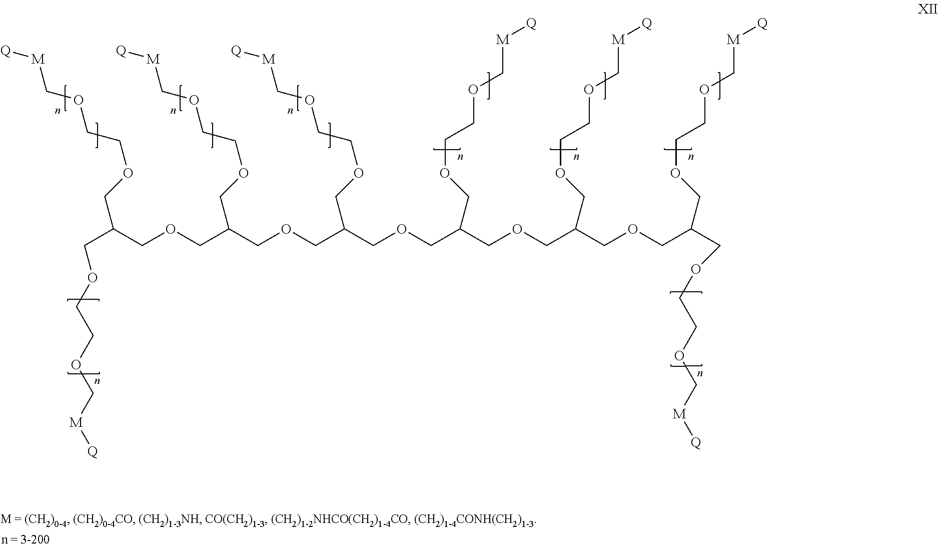

wherein FG is:
1. OH, SH, NH₂, OR², SR², COOH, COR², etc.
2. Maleimido, azido, NHS ester, hydrazido, izocyano and etc. groups.
3. Fluorescein, rhodamine, Cy3, Cy5, Cy7, Cy7.5 dyes, etc.
4. Biotinyl
5. Chelating group selected from the group consisting of a radical of DOTA, NOTA, TETA, DOTANGA etc. with radioactive metal for PET.
6. The therapeutic agent (cell-killing molecular agent such as tubulysin)
   wherein Q is head-group 1-6 from Scheme 1.

Scheme 3. Several head-group compounds that have been synthesized, used for attachment to linkers, and tested in vitro.

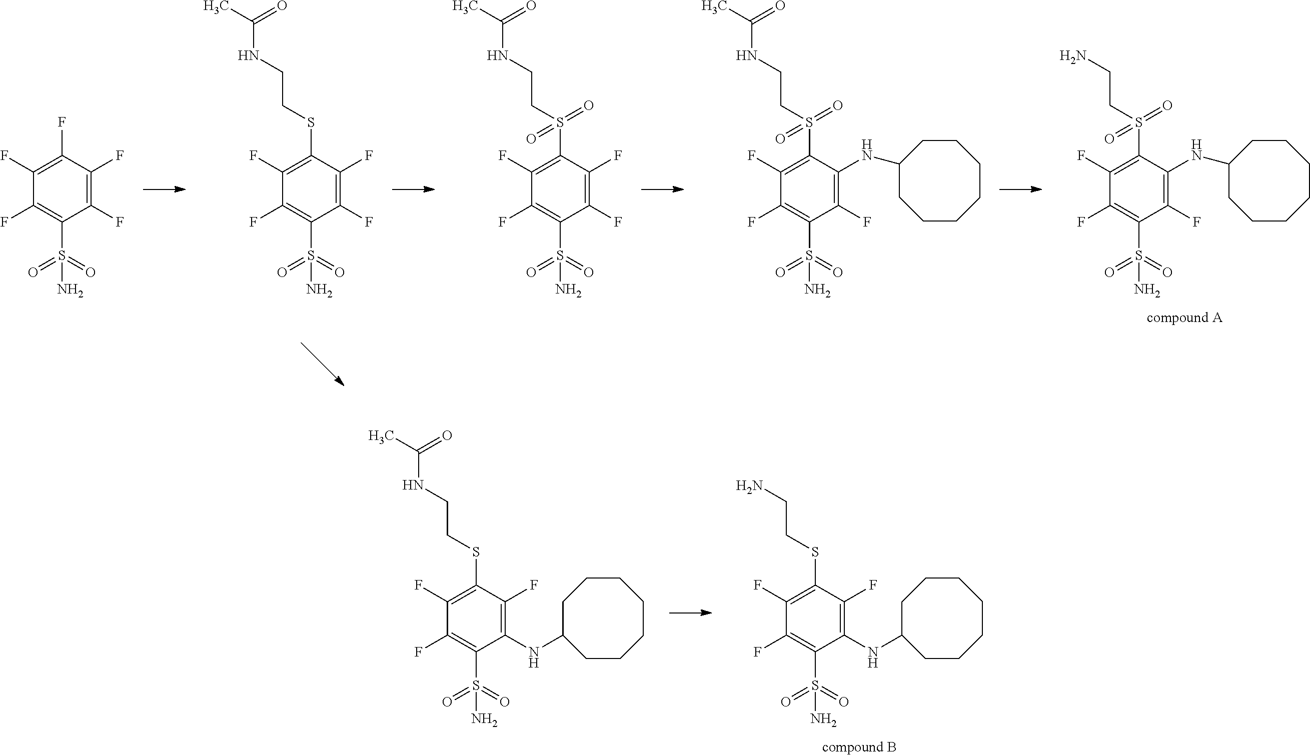

Fluorescent Thermal Shift Assay Experiments

Experiments were carried out in a Corbett Rotor-Gene 6000 (QIAGEN Rotor-Gene Q) instrument using the blue channel (365±20 nm excitation and 460±15 nm detection). The protein solution in the absence and presence of various compound concentrations was heated from 25 to 99° C. (heating rate 1° C./min). The melting temperature Tₘ shift was determined by following the fluorescence of 8-anilino-1-naphthalene sulfonate (ANS). The samples consisted of 5 μM protein (except 10 μM for CA IV), different concentrations of compound (usually 0-200 μM), 50 μM ANS and 50 mM sodium phosphate buffer (at pH 7.0) containing 100 mM sodium chloride and 2% (v/v) of DMSO. Compound binding constant was obtained from protein Tₘ as a function of the added ligand concentration. Data analysis was performed, and the curves were fit so that the binding constant is determined for 37° C.

Determination of the Inhibition of CA Enzymatic Activity

The inhibition of hydratase activity of carbonic anhydrase I, II, and IX was performed using Applied Photophysics SX.18 MV-R instrument at 24° C. Theaturated substrate solution was prepared by bubbling carbon dioxide gas into Milly-Q water for 1 hour at room temperature. Phenol red was used as a pH indicator to follow the absorbance (A-557 nm) while CA acidified the medium. The samples consisted of 300 nM CA I, 50 nM CA II, 20 nM or 100 nM CA IX, and 0-10 μM AZ19-3-2 or EA20-1 (≤0.3% DMSO), 30 μM phenol red, 25 mM Hepes buffer (at pH 7.5) containing 0.2 M sodium sulfate. Raw curves were fitted using a single exponential model, and the dissociation constants were determined using the Morrison equation:

$$CA act . ( \% ) = ( 1 - \frac{( [ C A ] + [ I ] + K d - \sqrt{{( [ C A ] + [ I ] + K d )}^{2} - 4 [ C A ] [ I ]}}{2 [ C A ]} ) \cdot 100 \%$$

Where [CA] is a total added concentration of active CA, [I]—total added inhibitor concentration, and K_{d} is inhibitor binding affinity.

### EMBODIMENTS OF THE INVENTION

### Example 1

### N-[2-[2-(cyclooctylamino)-3,5,6-trifluoro-4-sulfamoyl-phenyl]sulfonylethyl]-3-[2-[2-[2-[2-[2-[2-[2-[2-[2-[2-[2-[2-[3-[2-[2-(cyclooctylamino)-3,5,6-trifluoro-4-sulfamoyl-phenyl]sulfonylethylamino]-3-oxo-propoxy]ethoxy]ethoxy]ethoxy]ethoxy]ethoxy]ethoxy]ethoxy]ethoxy]ethoxy]ethoxy]ethoxy]eth oxy]propanamide (AZ19-3-2)

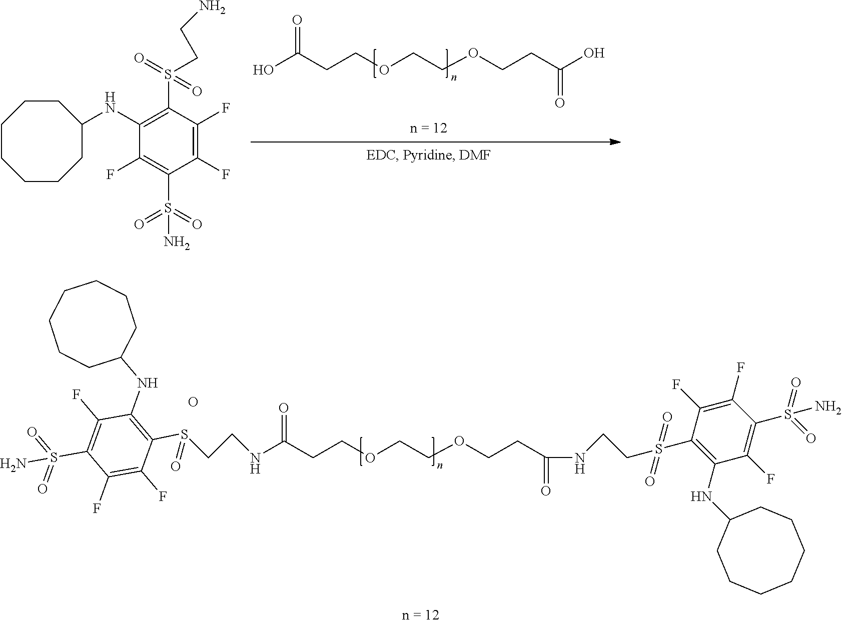

The mixture of O,O′-bis(2-carboxyethyl)dodecaethylene glycol (0.095 g, 0.137 mmol), N-ethyl-N′-(3-dimethylaminopropyl)carbodiimide hydrochloride (0.058 g, 0.302 mmol), 4-(2-aminoethylsulfonyl)-3-(cyclooctylamino)-2,5,6-trifluoro-benzenesulfonamide hydrochloride (0.145 g, 0.300 mmol), pyridine (0.200 mL, 2.476 mmol), and DMF (2.0 mL) was stirred at 20° C. for 48 h. The mixture was diluted with brine (20 mL) and extracted with EtOAc (3×10 mL).

The combined organic phase was dried over MgSO₄ and evaporated in reduced pressure. The product was purified by chromatography on a silica gel column (0.040-0.063 mm) with MeOH:CHCl₃ (1:10), Rf=0.55. Yield: 0.078 g, 37%, brownish oily residue.

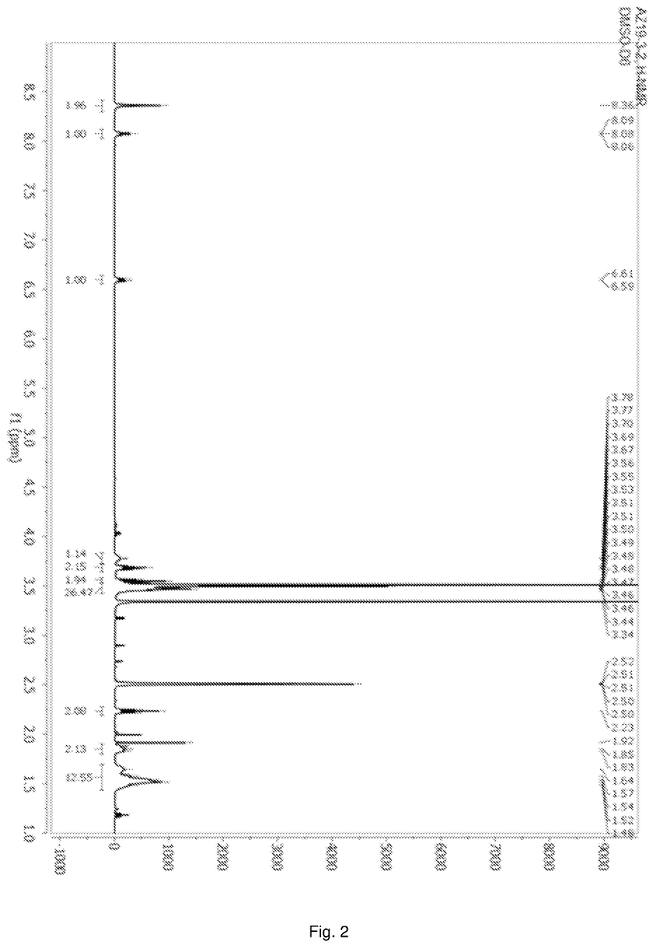

¹H NMR (400 MHz, DMSO-D₆) (shown in FIG. 2): 1.36-1.59 (20H, m, cyclooctane), 1.61-1.69 (4H, m, cyclooctane), 1.76-1.90 (4H, m, cyclooctane), 2.23 (4H, t, J=6.8 Hz, CH₂CO), 3.44-3.51 (52H, m, CH₂O), 3.55 (4H, t, J=6.4 Hz, CH₂N), 3.69 (4H, t, J=6.4 Hz, CH₂SO₂), 3.74-3.82 (2H, m, CHN of cyclooctane), 6.60 (2H, d, J=8.0 Hz, NH), 8.08 (2H, t, J=5.6 Hz, CONH), 8.36 (4H, s, SO₂NH₂).

¹⁹F NMR (376 MHz, DMSO-D₆): −124.7 (C3-F, br s), −134.4 (C5-F, dd, ¹J=26.7 Hz, ²J=12.0 Hz), −150.5 (C6-F, dd, ¹J=26.7 Hz, ²J=6.4 Hz). HRMS for C₆2H₁₀₂F₆N₆O₂₃S₄[(M+H)⁺]: calc. 1541.5856, found 1541.5812.

### Example 2

### Bis-(N′-[2-[2-(cyclooctylamino)-3,5,6-trifluoro-4-sulfamoyl-phenyl]sulfonylethyl]butanediamide)-mPEG₂₀₀₀ (E20-1)

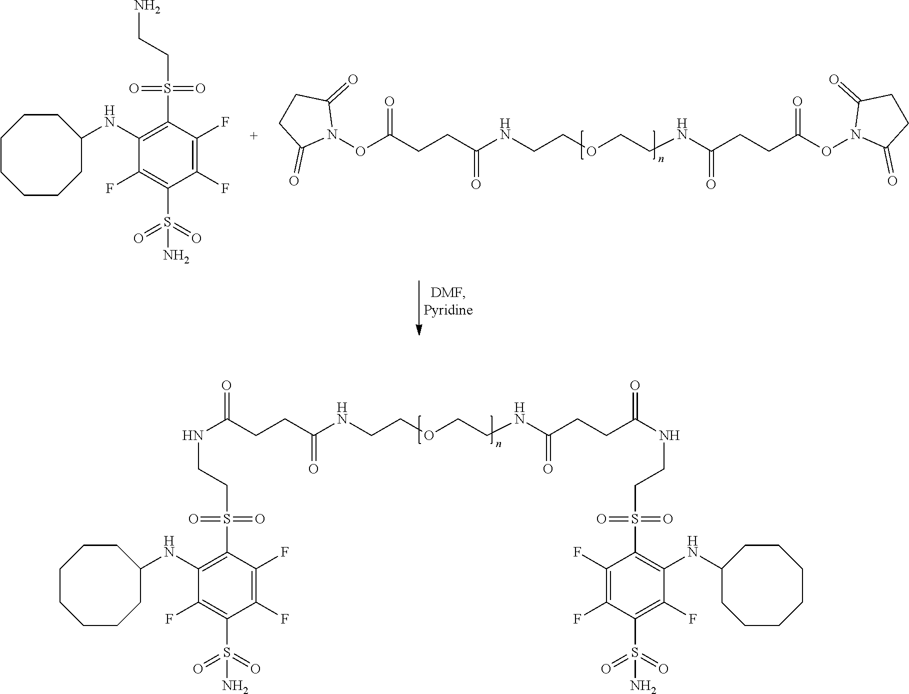

The mixture of O,O′-Bis[2-(N-Succinimidyl-succinylamino)ethyl]polyethylene glycol (average Mₙ 2000 g/mol, Sigma-Aldrich) (0.020 g, 0.010 mmol), 4-(2-aminoethylsulfonyl)-3-(cyclooctylamino)-2,5,6-trifluoro-benzenesulfonamide hydrochloride (0.012 g, 0.025 mmol), pyridine (0.050 mL), and DMF (0,500 mL) was stirred at 20° C. for one week. The mixture was diluted with brine (5 mL) and extracted with EtOAc (3×5 mL). The combined organic phase was dried over MgSO₄ and evaporated in reduced pressure. The product was purified by chromatography on a silica gel column (0.040-0.063 mm) with MeOH:CHCl₃(1:10), R_{f}=0.51. Yield: 0.012 g, 45%, brownish oily residue.

¹H NMR (400 MHz, DMSO-D₆): 1.44-1.59 (20H, m, cyclooctane), 1.61-1.68 (4H, m, cyclooctane), 1.82-1.87 (4H, m, cyclooctane), 2.18-2.30 (8H, m, CH₂CO), 3.17 (4H, q, J=6.0 Hz, CH₂NH), 3.44-3.51 (148H, m, CH₂O, CH₂N), 3.67 (4H, t, J=6.8 Hz, CH₂SO₂), 3.77 (2H, br s, CHN of cyclooctane), 6.59 (2H, d, J=8.8 Hz, NH), 7.90 (2H, t, J=5.6 Hz, CONH), 8.06 (2H, t, J=5.6 Hz, CONH), 8.24 (4H, s, SO₂NH₂).

¹⁹F NMR (376 MHz, DMSO-D₆): −124.7 (br s), −134.5 (dd, ¹J=26.7 Hz, ²J=11.3 Hz), −150.5 (dd, ¹J=26.7 Hz, ²J=6.0 Hz).

### Example 3

### 3-[2-[2-[2-[2-[2-[2-[2-[2-[2-[2-[2-[2-[3-[2-[2-(cyclooctylamino)-3,5,6-trifluoro-4- sulfamoyl-phenyl]sulfonylethylamino]-3-oxo-propoxy]ethoxy]ethoxy]ethoxy]ethoxy]ethoxy]ethoxy]ethoxy]ethoxy]ethoxy]ethoxy]ethoxy]eth oxy]propanoic acid (AZ19-3-1)

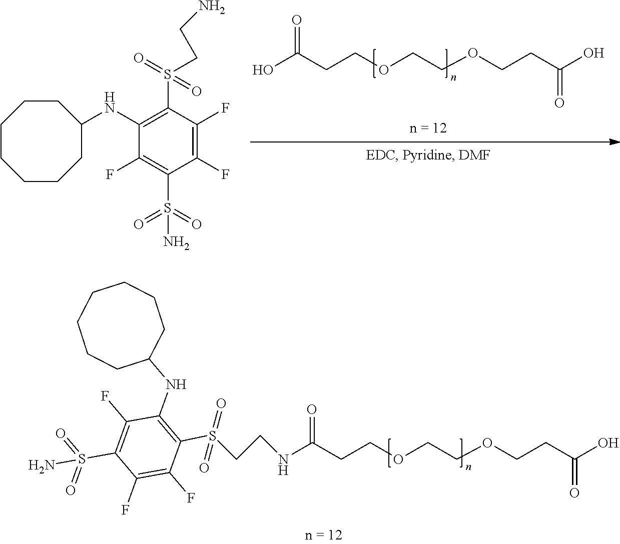

The mixture of O,O′-bis(2-carboxyethyl)dodecaethylene glycol (0.095 g, 0.137 mmol), N-ethyl-N′-(3-dimethylaminopropyl)carbodiimide hydrochloride (0.024 g, 0.151 mmol), 4-(2-aminoethylsulfonyl)-3-(cyclooctylamino)-2,5,6-trifluoro-benzenesulfonamide hydrochloride (0.072 g, 0.150 mmol), pyridine (0.100 mL, 1.238 mmol), and DMF (1.0 mL) was stirred at 20° C. for 48 h. The mixture was diluted with brine (20 mL) and extracted with EtOAc (3×10 mL). The combined organic phase was dried over MgSO₄ and evaporated in reduced pressure. The product was purified by chromatography on a column of silica gel (0.040-0.063 mm) with MeOH:CHCl₃ (1:10), Rf=0.59. Yield: 0.020 g, 13%, brownish oily residue.

¹H NMR (400 MHz, DMSO-D₆): 1.36-1.69 (12H, m, cyclooctane), 1.76-1.90 (2H, m, cyclooctane), 2.23 (2H, t, J=6.8 Hz, CH₂CO), 2.53 (2H, t, J=6.4 Hz, CH₂CO), 3.44-3.51 (52H, m, CH₂O), 3.55 (2H, t, J=6.4 Hz, CH₂N), 3.69 (2H, t, J=6.4 Hz, CH₂SO₂), 3.74-3.82 (1H, m, CHN of cyclooctane), 6.57 (1H, d, J=8.0 Hz, NH), 8.08 (1H, t, J=5.6 Hz, CONH), 8.36 (2H, s, SO₂NH₂), 11.99 (1H, br s, COOH)

¹⁹F NMR (376 MHz, DMSO-D₆): −124.7 (C3-F, br s), −134.4 (C5-F, dd, ¹J=26.7 Hz, ²J=12.0 Hz), −150.5 (C6-F, dd, ¹J=26.7 Hz, ²J=6.4 Hz).

### Example 4

### N′-[2-[2-(cyclooctylamino)-3,5,6-trifluoro-4-sulfamoyl-phenyl]sulfonylethyl]-N-(2-O-methyl-mPEG₇₅o)butanediamide (E20-2)

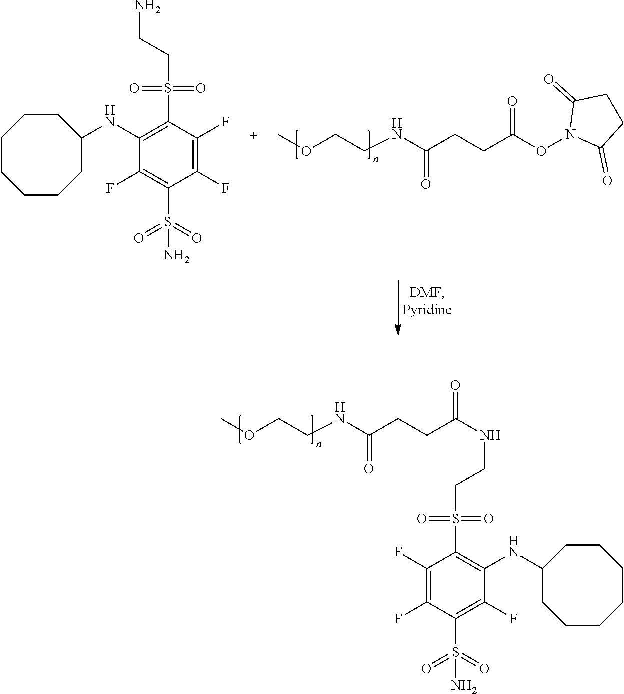

The mixture of O—[(N-Succinimidyl)succinyl-aminoethyl]-O′-methylpolyethylene glycol (average Mₙ 750 g/mol, Sigma-Aldrich) (0.033 g, 0.044 mmol), 4-(2-aminoethylsulfonyl)-3-(cyclooctylamino)-2,5,6-trifluoro-benzenesulfonamide hydrochloride (0.025 g, 0.053 mmol), pyridine (0.050 mL), and DMF (0,500 mL) was stirred at 20° C. for three weeks. The mixture was diluted with brine (5 mL) and extracted with EtOAc (3×5 mL). The combined organic phase was dried over MgSO₄ and evaporated in reduced pressure. The product was purified by chromatography on a column of silica gel (0.040-0.063 mm) with MeOH:CHCl₃ (1:7), R_{f}=0.65. Yield: 0.031 g, 65%, brownish oily residue.

¹H NMR (400 MHz, DMSO-D₆): 1.48-1.57 (10H, m, cyclooctane), 1.64 (2H, br s, cyclooctane), 1.82-1.89 (2H, m, cyclooctane), 2.18-2.29 (4H, m, CH₂CO), 3.17 (2H, q, J=4.7 Hz, CH₂NH), 3.24 (3H, s, OCH₃), 3.39-3.43 (2H, m, CH₂N), 3.44-3.51 (48H, m, CH₂O), 3.67 (2H, t, J=6.4 Hz, CH₂SO₂), 3.78 (1H, br s, CHN of cyclooctane), 6.59 (1H, d, J=8.4 Hz, NH), 7.89 (1H, t, J=5.6 Hz, CONH), 8.06 (1H, t, J=5.2 Hz, CONH), 8.39 (2H, s, SO₂NH₂). ¹⁹F NMR (376 MHz, DMSO-D₆): −124.7 (br s), −134.5 (dd, ¹J=26.3 Hz, ²J=11.3 Hz), −150.5 (dd, ¹J=26.3 Hz, ²J=3.8 Hz).

### Example 5

### 4arm-PEG5000-tetraacetamide, tetrakis-(N-[2-[2-(cyclooctylamino)-3,5,6-trifluoro-4-sulfamoyl-phenyl]sulfonylethyl]) (E20-3)

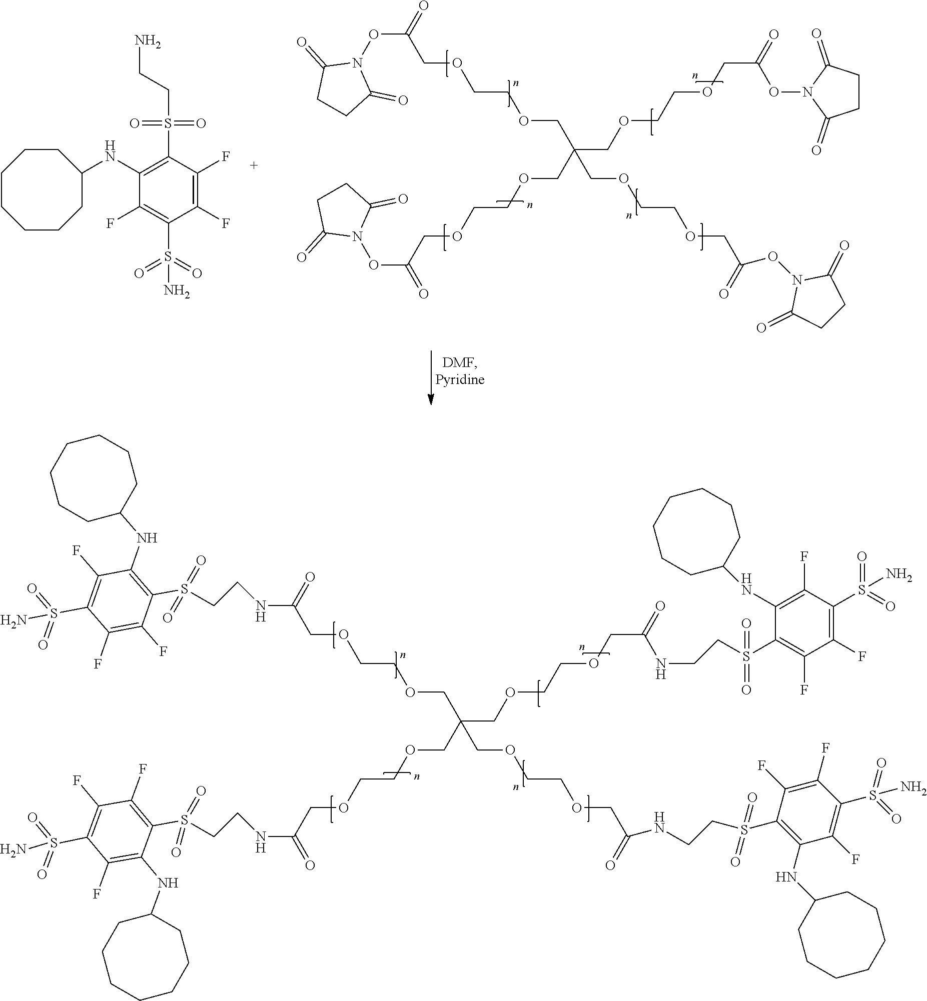

The mixture of 4arm-PEG5K-Succinimidyl Carboxymethyl Ester (average Mₙ 5000 g/mol, Sigma-Aldrich) (0.043 g, 0.0086 mmol), 4-(2-aminoethylsulfonyl)-3-(cyclooctylamino)-2,5,6-trifluoro-benzenesulfonamide hydrochloride (0.021 g, 0.043 mmol), pyridine (0.050 mL), and DMF (0,500 mL) was stirred at 20° C. for three weeks. The mixture was diluted with brine (5 mL) and extracted with EtOAc (3×5 mL). The combined organic phase was dried over MgSO₄ and evaporated in reduced pressure. The product was purified by chromatography on a column of silica gel (0.040-0.063 mm) with MeOH:CHCl₃ (1:7), R_{f}=0.23. Yield: 0.054 g, 46%, brownish oily residue.

¹H NMR (400 MHz, DMSO-D₆): 1.48-1.57 (40H, m, cyclooctane), 1.63-1.65 (8H, m, cyclooctane), 1.82-1.88 (8H, m, cyclooctane), 2,59 (8H, s, OCH₂C), 3.51-3.54 (392H, m, CH₂O, CH₂N), 3.67-3.69 (4H, m, CHN of cyclooctane), 3.75 (8H, t, J=6.8 Hz, CH₂SO₂), 3.80 (8H, s, COCH₂O), 6.61 (4H, d, J=8.0 Hz, NH), 7.86 (4H, t, J=5.2 Hz, CONH), 8.38 (8H, br s, SO₂NH₂).

¹⁹F NMR (376 MHz, DMSO-D₆): −124.8 (br s), −134.2 (dd, ¹J=26.3 Hz, ²J=11.3 Hz), −150.7 (dd, ¹J=26.3 Hz, ²J=3.8 Hz).

### Example 6

### Bis(3-aminopropyl)PEG1500, N,N′-bis(4-[(4-cyclohexylsulfanyl-3-sulfamoyl-benzoyl)amino]butanoyl) (LS20-1-5)

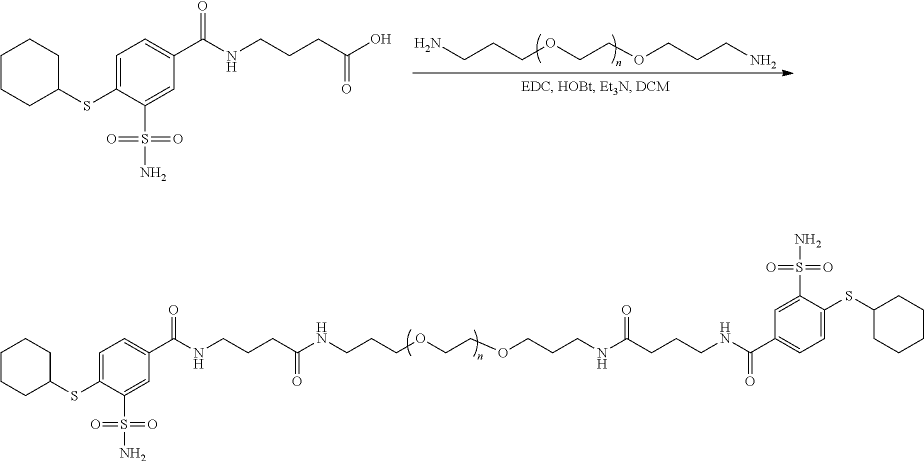

4-(4-(cyclohexylthio)-3-sulfamoylbenzamido)butanoic acid (0.050 g, 0.125 mmol) was dissolved in the minimum amount of anhydrous CH₂Cl₂ and, while stirring, each of HOBt (0.017 g, 0.125 mmol), EDC (0.024 g, 0.125 mmol) and TEA (0.017 ml, 0.125 mmol) were added in this order. To the resulting solution was added poly(ethylene glycol)bis(3-aminopropyl) terminated (average Mₙ=1500 g/mol, Sigma-Aldrich) (0.047 g, 0.031 mmol). The reaction was left stirring at room temperature for three weeks. After removing the solvent, the product was purified by Dry Column Vacuum Chromatography on a column of silica gel (15-40 μm) with EtOAc/MeOH gradient. Yield: 0.023 g, 33%, brownish oily residue.

¹H BMR (400 MHz, DMSO-d₆, d): 1.18-1.80 (24H, m, cyclohexane and CONHCH₂CH₂CH₂O), 1.91-1.98 (4H, m, CONHCH₂CH₂CH₂CONH), 2.10 (4H, t, ³J=7.4 Hz, CONHCH₂CH₂CH₂CONH), 3.06 (4H, m, CONHCH₂CH₂CH₂O), 3.25 (4H, q, ³J=5.9 Hz, CONHCH₂CH₂CH₂CONH), 3.49-3.51 (274H, m, OCH₂CH₂O and CONHCH₂CH₂CH₂O), 3.65-3.69 (2H, m, SCH), 7.30 (4H, br s, SO₂NH₂) 7.66 (2H, d, ³J=8.4 Hz, Ar—H), 7.92 (2H, m, CONHCH₂CH₂CH₂CONH), 7.95 (2H, dd, ³J=8.2 Hz, ⁴J=2.3 Hz, Ar—H), 8.39 (2H, d, ⁴J=1.9 Hz, Ar—H), 8.83 (2H, t, ³J=5.4 Hz, CONHCH₂CH₂CH₂CONH).

### Example 7

### Bis(3-aminopropyl)PEG1500, N,N′-bis(4-cyclohexylsulfanyl-3-sulfamoyl-benzoyl) (LS20-2-5)

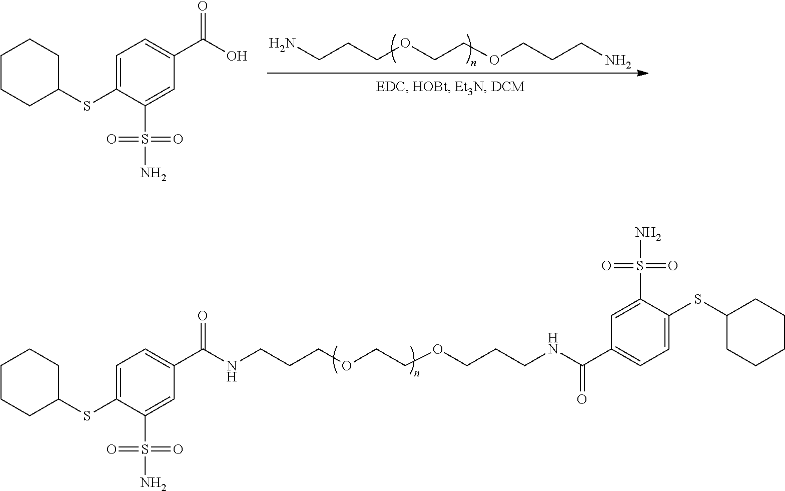

4-(cyclohexylthio)-3-sulfamoylbenzoic acid (0.050 g, 0.159 mmol) was dissolved in the minimum amount of anhydrous CH₂Cl₂ and, whilst stirring, each of HOBt (0.021 g, 0.159 mmol), EDC (0.030 g, 0.159 mmol) and TEA (0.022 ml, 0.159 mmol) were added in this order. To the resulting solution was added poly(ethylene glycol)bis(3-aminopropyl) terminated (average Mₙ=1500 g/mol, Sigma-Aldrich) (0.059 g, 0.04 mmol). The reaction was left stirring at room temperature for three weeks. After removing the solvent, the product was purified by Dry Column Vacuum Chromatography on a column of silica gel (15-40 μm) with EtOAc/MeOH gradient. Yield: 0.052 g, 63%, brownish oily residue.

¹H BMR (400 MHz, DMSO-d₆, 5): 1.19-1.98 (24H, m, cyclohexane and CONHCH₂CH₂CH₂O), 3.30 (4H, q, ³J=6.0 Hz, CONHCH₂CH₂CH₂O), 3.42 (4H, m, CONHCH₂CH₂CH₂O), 3.49-3.51 (196H, m, OCH₂CH₂O), 3.57 (2H, m, SCH), 7.36 (4H, br s, SO₂NH₂), 7.67 (2H, d, ³J=8.5 Hz, Ar—H), 7.94 (2H, dd, ³J=8.3 Hz, ⁴J=2.0 Hz, Ar—H), 8.37 (2H, d, ⁴J=2.0 Hz, Ar—H), 8.69 (2H, t, ³J=5.5 Hz, CONH).

### Example 8

The following compounds have been designed based on the syntheses described above

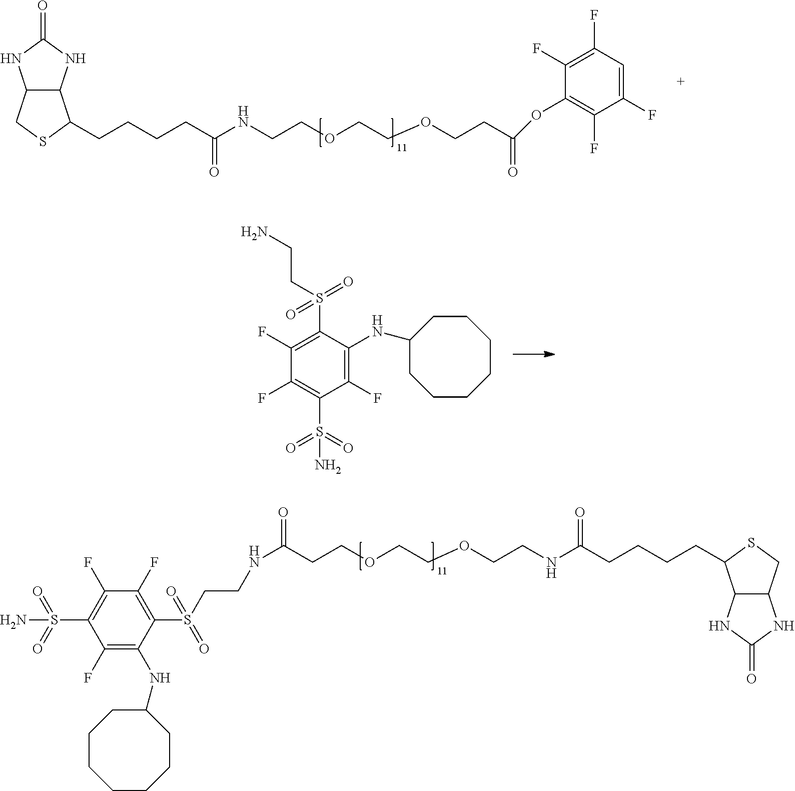

### N-[2-[2-[2-[2-[2-[2-[2-[2-[2-[2-[2-[2-[3-[2-[2-(cyclooctylamino)-3,5,6-trifluoro-4-sulfamoyl- phenyl]sulfonylmethylamine]-3-oxo-propoxy]ethoxy]ethoxy]ethoxy]ethoxy]ethoxy]ethoxy]ethoxy]ethoxy]ethoxy]ethoxy]ethoxy]ethyl]-5-(2-oxo-1,3,3a,4,6,6a-hexahydrothieno[3,4-d]imidazol-4-yl)pentanamide (AZ19-1)

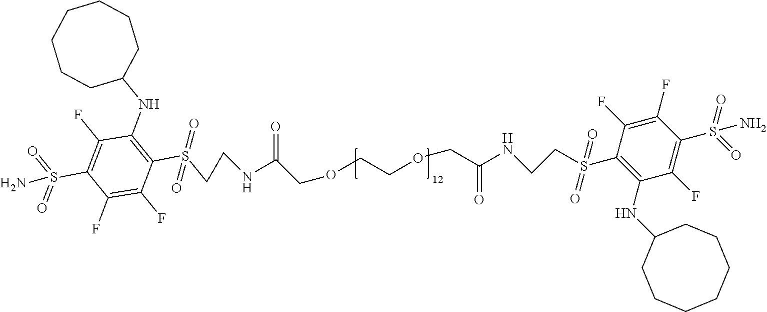

### PEG12-diacetamide, Bis(N-[2-[2-(cyclooctylamino)-3,5,6-trifluoro-4-sulfamoyl-phenyl]sulfonylethyl])

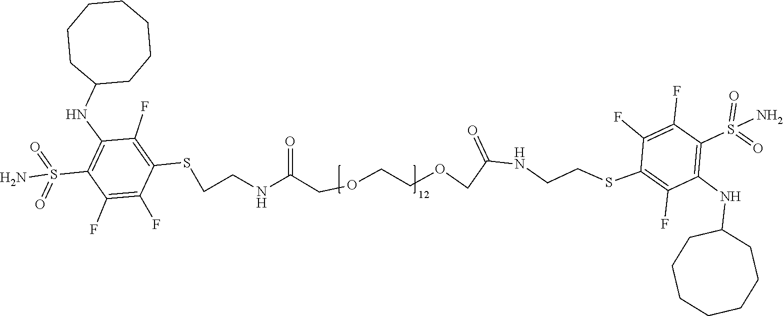

### PEG12-diacetamide, Bis(N-[2-[3-(cyclooctylamino)-2,5,6-trifluoro-4-sulfamoyl-phenyl]sulfanylethyl])

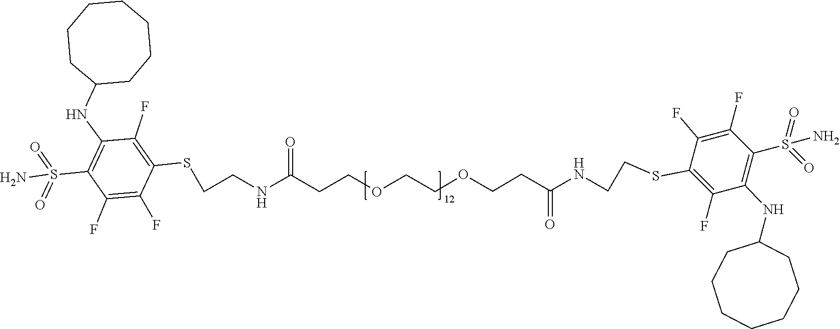

### PEG12-dipropanamide, Bis(N-[2-[3-(cyclooctylamino)-2,5,6-trifluoro-4-sulfamoyl-phenyl]sulfanylethyl])

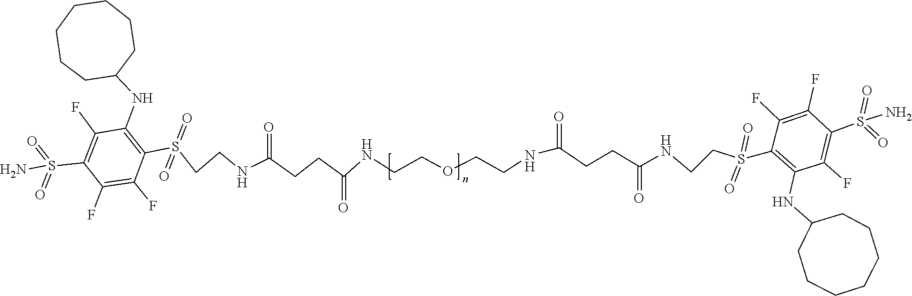

### Bis[2-[2-(cyclooctylamino)-3,5,6-trifluoro-4-sulfamoyl-phenyl]sulfonylethylamino]-PEG2000

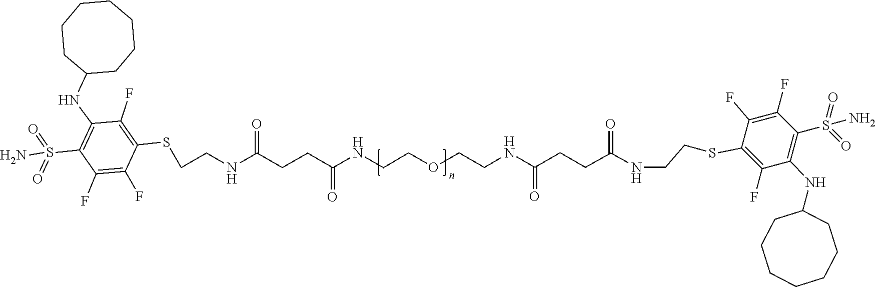

### Bis[2-[3-(cyclooctylamino)-2,5,6-trifluoro-4-sulfamoyl-phenyl]sulfanylethylamino]-PEG2000

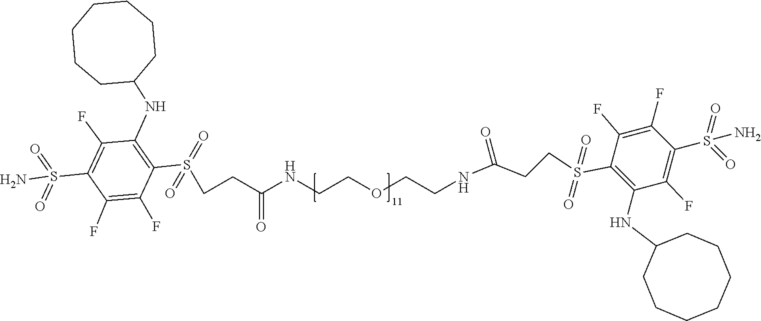

### N,N′-bis(3-[2-(cyclooctylamino)-3,5,6-trifluoro-4-sulfamoyl-phenyl]sulfonyl-propanoyl)-PEG12 diamine

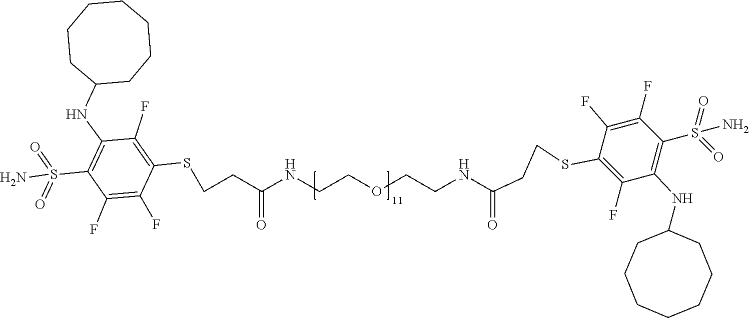

### N,N′-bis(3-[3-(cyclooctylamino)-2,5,6-trifluoro-4-sulfamoyl-phenyl]sulfanyl-propanoyl)-PEG12 diamine

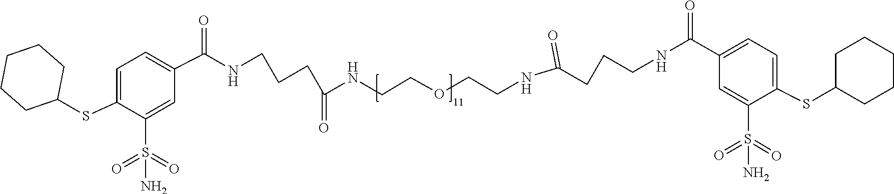

### N,N′-bis(4-[(4-cyclohexylsulfanyl-3-sulfamoyl-benzoyl)amino]butanoyl)-PEG12 diamine

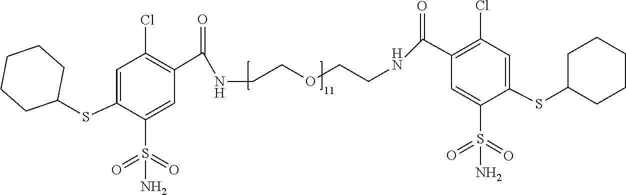

### N,N′-Bis(2-chloro-4-cyclohexylsulfanyl-5-sulfamoyl-benzoyl)-PEG12 diamine

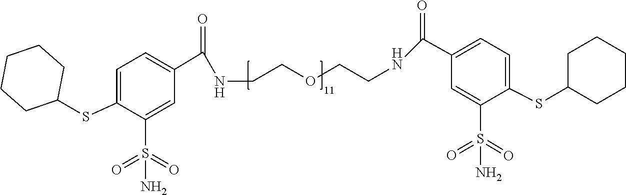

### N,N′-Bis(4-cyclohexylsulfanyl-3-sulfamoyl-benzoyl)-PEG12 diamine

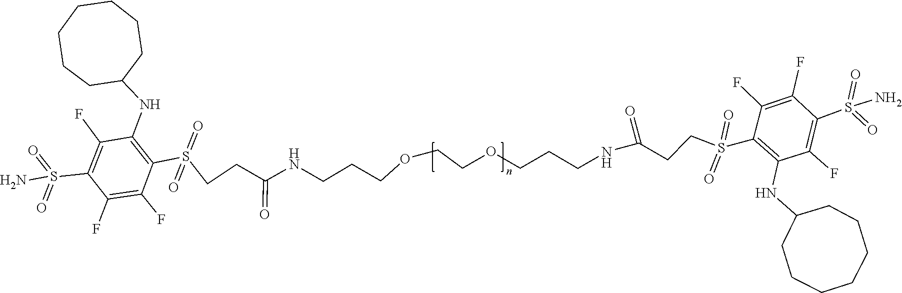

### Bis(3-aminopropyl)PEG1500, N,N′-bis(3-[2-(cyclooctylamino)-3,5,6-trifluoro-4-sulfamoyl-phenyl]sulfonyl-propanoyl)

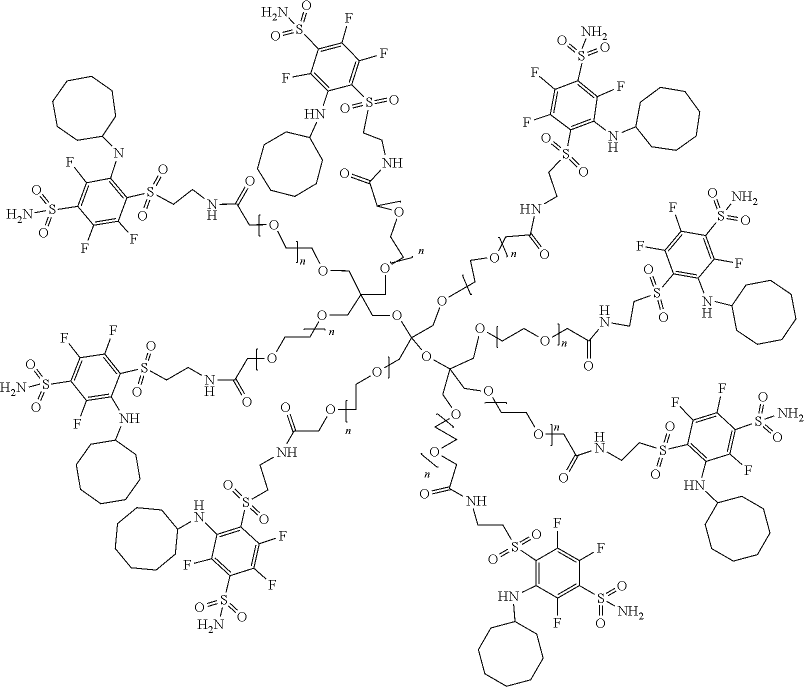

### 8arm-PEG10000-octaacetamide, octakis-(N-[2-[2-(cyclooctylamino)-3,5,6-trifluoro-4-sulfamoyl-phenyl]sulfonylethyl])

TABLE 1
Dissociation constants of compound binding to twelve catalytically active human recombinant CA isoforms as determined by
the fluorescent thermal shift assay (FTSA, 37° C., pH 7.0). Acetazolamide (AZM) has been used as a control. The VD11-4-2
compound is previously described as a head-group compound. The compound AZ19-3-1 bears one VD11-4-2 head-group
and a PEG linker. The compound AZ19-3-2 bears two VD11-4-2 head-groups and the PEG linker. The AZ19-1 compound
bears VD11-4-2 compound on one end and biotin on the other end of the PEG linker.

| | K_{d}, nM | | | | | | | | | | | |
|---|---|---|---|---|---|---|---|---|---|---|---|---|
| Compound | CA I | CA II | CA III | CA IV | CA VA | CA VB | CA VI | CA VII | CA IX | CA XII | CA XIII | CA XIV |
| AZ19-3-1 | 10000 | 250 | ≥200000 | 1000 | 10000 | 70 | 500 | 50 | 0.050 | 20 | 20 | 10 |
| AZ19-3-2 | 3300 | 100 | ≥200000 | 250 | 3000 | 70 | 330 | 330 | 0.015 | 20 | 2.0 | 8.3 |
| AZ19-1 | 10000 | 450 | ≥200000 | 1000 | 7100 | 83 | 980 | 150 | 0.10 | 50 | 50 | 27 |
| VD11-4-2 | 830 | 56 | 34000 | 61 | 3300 | 16 | 67 | 8.6 | 0.032 | 2.9 | 4.0 | 4.3 |
| E20-1 | 8200 | 400 | ≥200000 | 120 | 8300 | 39 | 270 | 48 | 0.004 | 39 | 20 | 49 |
| E20-2 | 8500 | 350 | ≥200000 | 820 | 8200 | 58 | 560 | 53 | 0.1 | 49 | 23 | 100 |
| E20-3 | 16000 | 1000 | >200000 | 810 | 2500 | 52 | 330 | 63 | 0.009 | 63 | 34 | 40 |
| LS20-1-5 | ≥200000 | ≥200000 | ≥200000 | ≥200000 | ≥200000 | ≥200000 | ≥200000 | ≥200000 | 1100 | ≥200000 | ≥200000 | >200000 |
| LS20-2-5 | ≥200000 | ≥200000 | ≥200000 | 23000 | ≥200000 | ≥200000 | >200000 | ≥200000 | 1900 | ≥200000 | ≥200000 | ≥200000 |
| AZM | 2400 | 46 | 40000 | 87 | 840 | 140 | 220 | 13 | 21 | 130 | 120 | 63 |

The compound AZ19-3-2 that bears two VD11-4-2 head-groups and the PEG linker bound to CA IX with similar affinities as a single head-group compound VD11-4-2. This is expected since the protein molecules were recombinantly made to be free in solution.

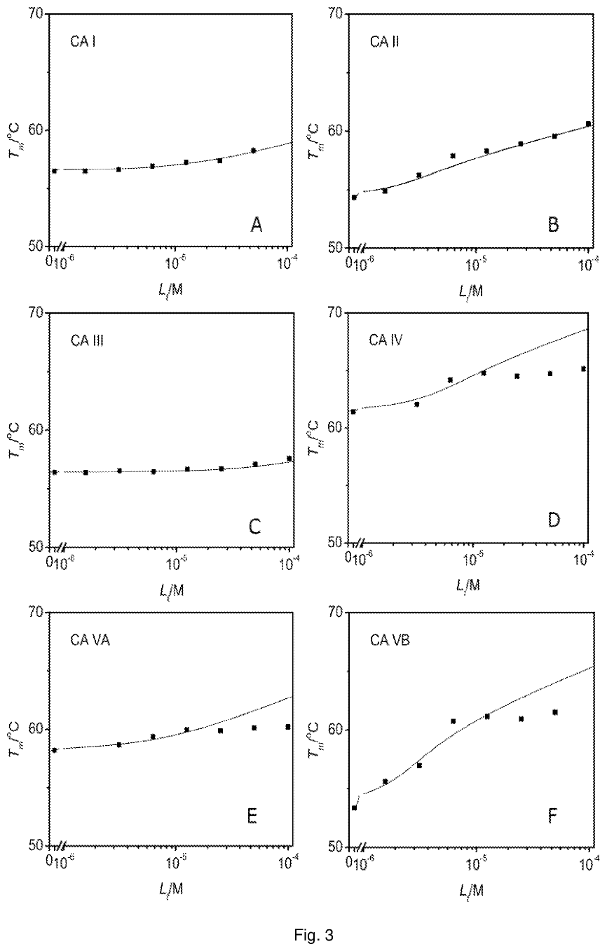

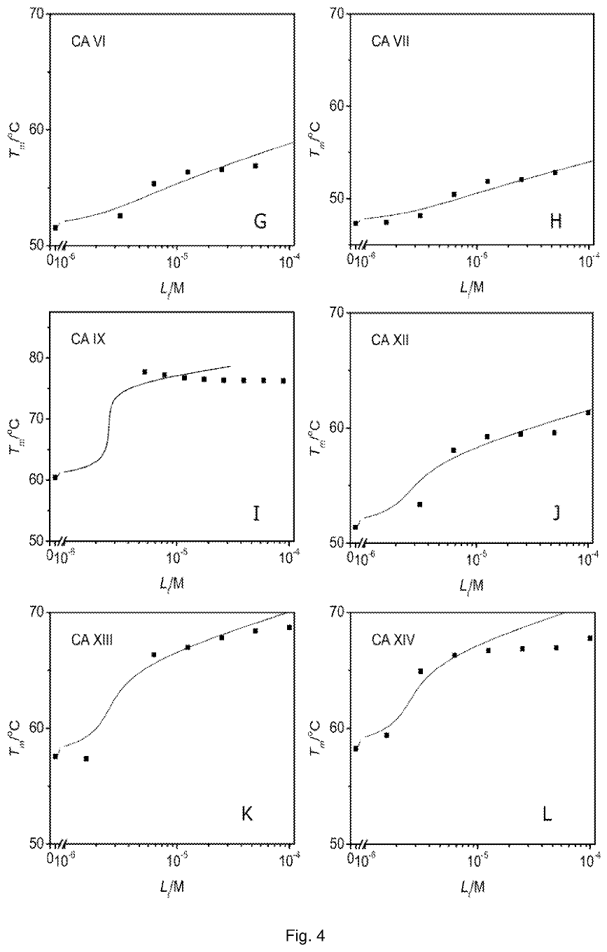

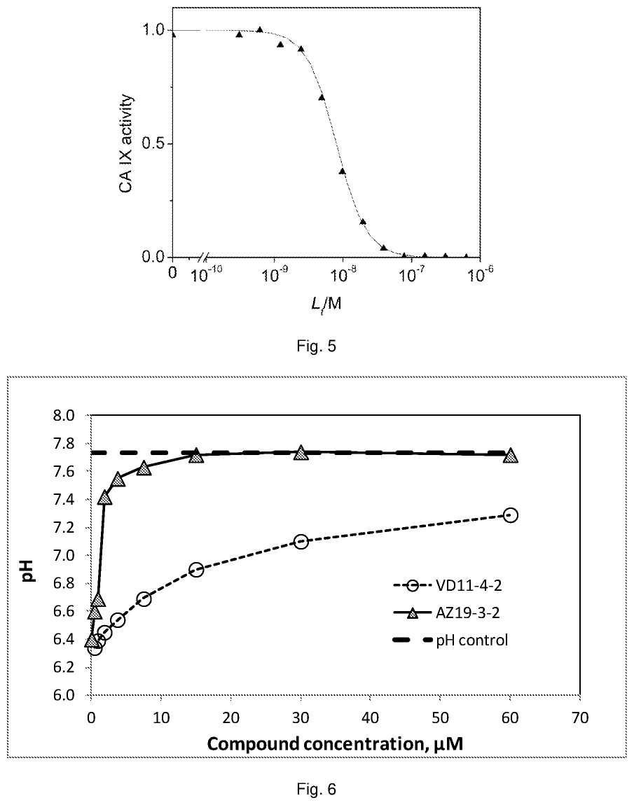

It is clear from the data in Table 1 and FIGS. 3 and 4 that the compounds where the head-group is attached to a linker such as PEG bind to carbonic anhydrases with essentially the same affinity as the head-group compounds themselves. The attached linker does not affect the affinity in a significant fashion. Furthermore, the double-headed compound also binds with the same affinity. The only difference is that it binds to two molecules of the enzyme and efficiently connects them in solution.

However, the main advantage and difference of the double-headed compounds becomes apparent in cell cultures, where, for example, the AZ19-3-2 compound that contains two head-groups and a PEG linker has been demonstrated to be at least 40-fold more efficient in stopping the effect of acidification of extracellular space (FIG. 6) and in the effect of inhibiting the growth of cancer cells. The compound AZ19-3-2 fully inhibited the acidification effect mainly caused by CA IX at 10 μM concentration. Even at 1 μM, the acidification was mostly inhibited. This effect was much stronger than the effect of the head-group compound itself (VD11-4-2), where even at 60 μM, the acidification effect was reduced by only approximately ⅔.

The main advantage of the multi-headed compounds is expected from the significantly increased affinity for membrane-attached CAs as compared with single-headed compounds. This is because when a multi-headed compound can reach several membrane-attached CAs simultaneously, the affinity is expected to be increased tremendously. It is known that the head-group compound VD11-4-2 has a residence half-time on CA IX equal to approximately 5 hours. In the case of a double-headed compound, when one head-group unbinds, the other one would remain bound, and before the second one unbinds, the first one is most likely to rebind again. Therefore, the affinity of such a double-headed compound would be much greater than of a single-headed compound. Note that it was impossible to directly demonstrate this increase in affinity for CA IX because the CA IX is prepared in solution in the form of free monomers. Therefore, in the in vitro experiment the compound would independently catch two CA IX molecules with the same affinity. The additive effect would only be visible in the cellular environment where CA IX is attached to the cell surface. A confirmation of the increase in effect was obtained only indirectly via the acidification experiment (FIG. 6).

There are also other potential mechanisms how the double or multi-headed compounds could have an advantage over single-headed ones. One possibility is that the multi-headed compounds could bind to multiple CA IX molecules and inhibit their dynamic mobility on the cell surface. The binding of other potential proteins-partners could be prevented via such a large linker-connected compound.

The above-discussed multi-headed compounds are significantly more effective for the inhibition of the acidification effect caused by CA IX. They thus could be applied for the diagnostics/visualization and treatment of various cancers where the expression of CA IX is overexpressed, such as but not limited to cervix carcinoma, esophageal carcinoma, pancreatic tumor, kidney carcinoma, endometrial adenocarcinoma, ovarian tumor, urinary bladder carcinoma, colonadenocarcinoma, lung tumor, liver carcinoma and breast adenocarcinoma or other diseases, such as glaucoma, epilepsy, high altitude sickness, or even neurodegenerative diseases such as Alzheimer's disease.

### REFERENCES

(1) The Carbonic Anhydrases; Dodgson, S. J., Tashian, R. E., Gros, G., Carter, N. D., Eds.; Springer US: Boston, M A, 1991. https://doi.org/10.1007/978-1-4899-0750-9.
(2) The Carbonic Anhydrases; Chegwidden, W. R., Carter, N. D., Edwards, Y. H., Eds.; Birkhä user Basel: Basel, 2000. https://doi.org/10.1007/978-3-0348-8446-4.
(3) Supuran, C. T.; Scozzafava, A.; Conway, J. Carbonic Anhydrase: Its Inhibitors and Activators; CRC Press, 2004; Vol. 1.
(4) Carbonic Anhydrase: Mechanism, Regulation, Links to Disease, and Industrial Applications; Frost, S. C., McKenna, R., Eds.; Subcellular Biochemistry; Springer Netherlands: Dordrecht, 2014; Vol. 75. https://doi.org/10.1007/978-94-007-7359-2.
(5) Matulis, D. CARBONIC ANHYDRASE AS DRUG TARGET: Thermodynamics and Structure Of; SPRINGER NATURE: S. I., 2019.
(6) Dudutienė, V.; Matulienė, J.; Smirnov, A.; Timm, D. D.; Zubrienė, A.; Baranauskienė, L.; Morkūnaite, V.; Smirnovienė, J.; Michailovienė, V.; Juozapaitienė, V.; Mickevic̆iūtė, A.; Kazokaitė, J.; Baks̆ytė, S.; Kasiliauskaitė, A.; Jachno, J.; Revuckienė, J.; Kis̆onaitė, M.; Pilipuitytė, V.; Ivanauskaitė, E.; Milinavic̆iūtė, G.; Smirnovas, V.; Petrikaitė, V.; Kairys, V.; Petrauskas, V.; Norvais̆ as, P.; Lingė, D.; Gibiez̆a, P.; Capkauskaitė, E.; Zaks̆auskas, A.; Kazlauskas, E.; Manakova, E.; Graz̆ulis, S.; Ladbury, J. E.; Matulis, D. Discovery and Characterization of Novel Selective Inhibitors of Carbonic Anhydrase IX. J. Med. Chem. 2014, 57(22), 9435-9446. https://doi.org/10.1021/jm501003k.

(7) Linkuvienė, V.; Zubrienė, A.; Manakova, E.; Petrauskas, V.; Baranauskienė, L.; Zaks̆auskas, A.; Smirnov, A.; Graz̆ulis, S.; Ladbury, J. E.; Matulis, D. Thermodynamic, Kinetic, and Structural Parameterization of Human Carbonic Anhydrase Interactions toward Enhanced Inhibitor Design. Q. Rev. Biophys. 2018, 51. https://doi.org/10.1017/S0033583518000082.

(8) Mahalingam, S. M.; Chu, H.; Liu, X.; Leamon, C. P.; Low, P. S. Carbonic Anhydrase IX-Targeted Near-Infrared Dye for Fluorescence Imaging of Hypoxic Tumors. Bioconjug. Chem. 2018. https://doi.org/10.1021/acs.bioconjchem.8b00509.
(9) Mahalingam, S. M.; Kularatne, S. A.; Myers, C. H.; Gagare, P.; Norshi, M.; Liu, X.; Singhal, S.; Low, P. S. Evaluation of Novel Tumor-Targeted Near-Infrared Probe for Fluorescence-Guided Surgery of Cancer. J. Med. Chem. 2018. https://doi.org/10.1021/acs.jmedchem.8b01115.
(10) Marks, I. S.; Gardeen, S. S.; Kurdziel, S. J.; Nicolaou, S. T.; Woods, J. E.; Kularatne, S. A.; Low, P. S. Development of a Small Molecule Tubulysin B Conjugate for Treatment of Carbonic Anhydrase IX Receptor Expressing Cancers. Mol. Pharm. 2018. https://doi.org/10.1021/acs.molpharmaceut.8b00139.

1. A conjugate of formula Q-L-FG, wherein Q is a binding ligand of carbonic anhydrase IX, L is an optional linker, and FG is a functional group, therapeutical agent, or an imaging agent.

2. A conjugate according to claim 1, wherein the CA IX ligand Q is selected from the group consisting of 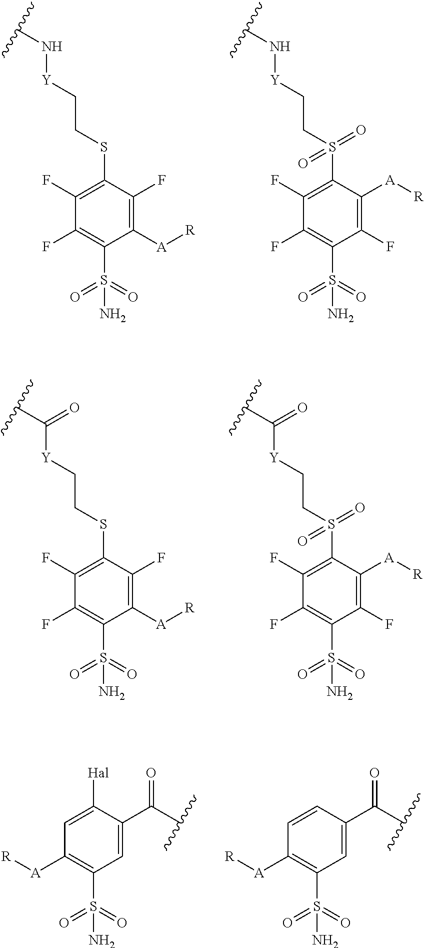
where
A is NH or S
Y is (NH)₀₋₁, (CH₂)₀₋₁₂, (OCH₂CH₂)₀₋₁₂ or (NHCH₂CH₂)₀₋₄
Hal is Cl or Br
R is alkyl, aryl, cycloalkyl, (CH₂)₁₋₄cycloalkyl or (CH₂)₁₋₄aryl.

3. A conjugate according to claim 1, wherein the optional linker L is of the formula 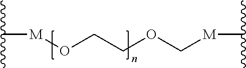
where
each M is independently selected from the group consisting of (CH₂)₀₋₄, (CH₂)₀₋₄CO, (CH₂)₁₋₃NH, CO(CH₂)₁₋₃, (CH₂)₁₋₂NHCO(CH₂)₁₋₄CO, and (CH₂)₁₋₄CONH(CH₂)₁₋₃.
and n is from 3 to 200

4. A conjugate according to claim 1, wherein FG is a functional group, therapeutical agent or an imaging agent selected from:
OH, SH, NH2, COOH, N3, NHNH2, maleimido, NHS ester, izocyanato, and izothiocyanato, fluorescein, rhodamine, Cy3, Cy5, Cy7, Cy7.5 dyes,
biotinyl,
chelating group selected from the group consisting of a radical of DOTA, NOTA, TETA, DOTANGA with radioactive metal for PET,
therapeutic agent.

5. A compound according to claim 1 which is:
3-[2-[2-[2-[2-[2-[2-[2-[2-[2-[2-[2-[2-[3-[2-[2-(cyclooctylamino)-3,5,6-trifluoro-4- sulfamoyl-phenyl]sulfonylethylamino]-3-oxo-propoxy]ethoxy]ethoxy]ethoxy]ethoxy]ethoxy]ethoxy]ethoxy]ethoxy]ethoxy]ethoxy]ethoxy]ethoxy] propanoic acid;
N′-[2-[2-(cyclooctylamino)-3,5,6-trifluoro-4-sulfamoyl-phenyl]sulfonylethyl]-N-(2-O-methyl-mPEG750)butanediamide;
N-[2-[2-[2-[2-[2-[2-[2-[2-[2-[2-[2-[2-[3-[2-[2-(cyclooctylamino)-3,5,6-trifluoro-4-sulfamoyl- phenyl]sulfonylethylamino]-3-oxo-propoxy]ethoxy]ethoxy]ethoxy]ethoxy]ethoxy]ethoxy]ethoxy]ethoxy]ethoxy]ethoxy]ethoxy]ethyl]-5-(2-oxo-1,3,3a,4,6,6a-hexahydrothieno[3,4-d]imidazol-4-yl)pentanamide.

6. A pharmaceutical composition comprising a compound according to claim 1 and pharmaceutically acceptable diluents, excipients or carriers.

7. A pharmaceutical composition according to claim 6 for use in the treatment of various cancers selected from the group of cervix carcinoma, esophageal carcinoma, pancreatic tumor, kidney carcinoma, endometrial adenocarcinoma, ovarian tumor, urinary bladder carcinoma, colon adenocarcinoma, lung tumor, liver carcinoma, and breast adenocarcinoma.

8. A pharmaceutical composition according to claim 6 for use in the treatment of other diseases, such as glaucoma, epilepsy, high altitude sickness, or Alzheimer's disease.

9. A compound according to claim 5 used for tumor visualization, fluorescent probes for visualization, or PET for tumor visualization.

10. A conjugate of formula FG₀₋₁-L-Q₂₋₈, containing at least two Q, wherein Q is a binding ligand of carbonic anhydrase IX, L is an optional linker, and the linker is unsubstituted or substituted by one or more identical or different FG groups, which are selected from functional groups, therapeutical agents or an imaging agents.

11. A conjugate according to claim 10, wherein the CA IX ligand Q is selected from the group consisting of: 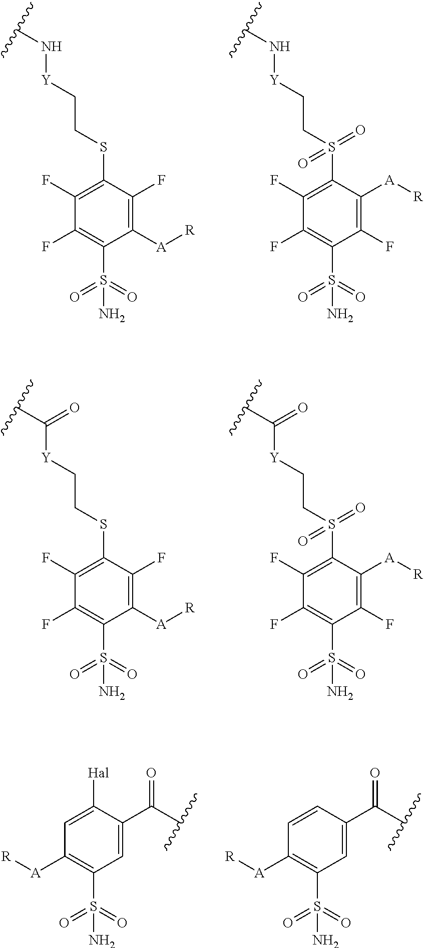
where
A is NH or S
Y is (NH)₀₋₁, (CH₂)₀₋₁₂, (OCH₂CH₂)₀₋₁₂ or (NHCH₂CH₂)₀₋₄
Hal is Cl or Br
R is alkyl, aryl, cycloalkyl, (CH₂)₁₋₄cycloalkyl or (CH₂)₁₋₄aryl.

12. A conjugate according to claim 10, wherein the optional linker L is is selected from the group consisting of 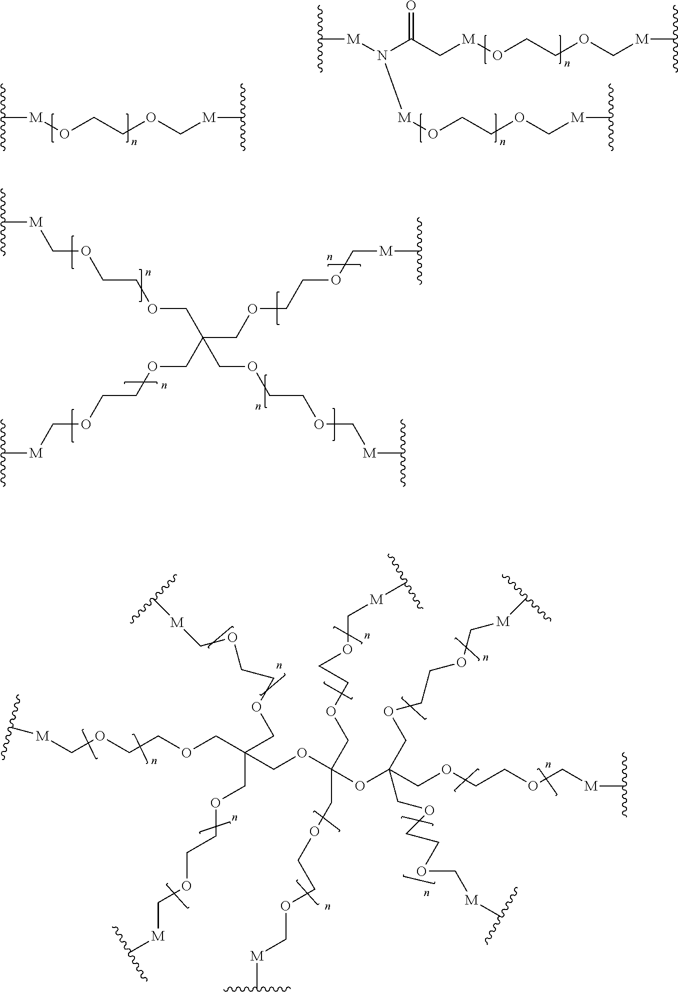 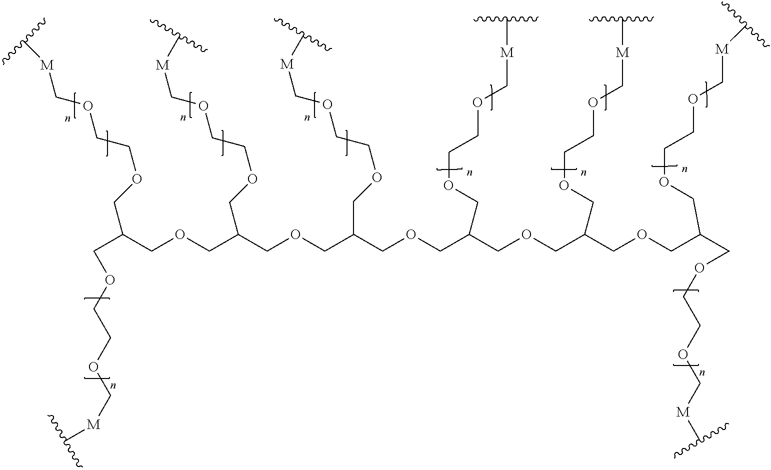
where
each M is independently selected from the group consisting of (CH₂)₀₋₄, (CH₂)₀₋₄CO, (CH₂)₁₋₃NH, CO(CH₂)₁₋₃, (CH₂)₁₋₂NHCO(CH₂)₁₋₄CO, (CH₂)₁₋₄CONH(CH₂)₁₋₃,
and n is from 8 to 200.

13. A conjugate according to claim 10, wherein FG is a functional group, therapeutical agent or an imaging agent selected from:
OH, SH, NH2, COOH, N3, NHNH2, maleimido, NHS ester, izocyanato, and izothiocyanato, fluorescein, rhodamine, Cy3, Cy5, Cy7, Cy7.5 dyes,
biotinyl,
chelating group selected from the group consisting of a radical of DOTA, NOTA, TETA, DOTANGA with radioactive metal for PET,
therapeutic agent.

14. A compound according to claim 10 which is:
N-[2-[2-(cyclooctylamino)-3,5,6-trifluoro-4-sulfamoyl-phenyl]sulfonylethyl]-3-[2-[2-[2-[2-[2-[2-[2-[2-[2-[2-[2-[2-[3-[2-[2-(cyclooctylamino)-3,5,6-trifluoro-4-sulfamoyl-phenyl]sulfonylethylamino]-3-oxo-propoxy]ethoxy]ethoxy]ethoxy]ethoxy]ethoxy]ethoxy]ethoxy]ethoxy]ethoxy]ethoxy]ethoxy]ethoxy] propanamide;
bis-(N′-[2-[2-(cyclooctylamino)-3,5,6-trifluoro-4-sulfamoyl-phenyl]sulfonylethyl]butanediamide)-mPEG₂₀₀₀;
PEG12-diacetamide, Bis(N-[2-[2-(cyclooctylamino)-3,5,6-trifluoro-4-sulfamoyl-phenyl]sulfonylethyl]);
PEG12-diacetamide, Bis(N-[2-[3-(cyclooctylamino)-2,5,6-trifluoro-4-sulfamoyl-phenyl]sulfanylethyl]);
PEG12-dipropanamide, Bis(N-[2-[2-(cyclooctylamino)-3,5,6-trifluoro-4-sulfamoyl-phenyl]);
PEG12-dipropanamide, Bis(N-[2-[3-(cyclooctylamino)-2,5,6-trifluoro-4-sulfamoyl-phenyl]sulfanylethyl]);
Bis[2-[2-(cyclooctylamino)-3,5,6-trifluoro-4-sulfamoyl-phenyl]sulfanylethylamino]-PEG2000;
Bis[2-[3-(cyclooctylamino)-2,5,6-trifluoro-4-sulfamoyl-phenyl]sulfanylethylamino]-PEG2000;
N,N′-bis(3-[2-(cyclooctylamino)-3,5,6-trifluoro-4-sulfamoyl-phenyl]sulfonyl-propanoyl)-PEG12 diamine;
N,N′-bis(3-[3-(cyclooctylamino)-2,5,6-trifluoro-4-sulfamoyl-phenyl]sulfonyl-propanoyl)-PEG12 diamine;
N,N′-bis(4-[(4-cyclohexylsulfanyl-3-sulfamoyl-benzoyl)amino]butanoyl)-PEG12 diamine;
N,N′-bis(2-chloro-4-cyclohexylsulfanyl-5-sulfamoyl-benzoyl)-PEG12 diamine;
N,N′-bis(4-cyclohexylsulfanyl-3-sulfamoyl-benzoyl)-PEG12 diamine;
Bis(3-aminopropyl)PEG1500, N,N′-bis(3-[2-(cyclooctylamino)-3,5,6-trifluoro-4-sulfamoyl-phenyl]sulfonyl-propanoyl);
Bis(3-aminopropyl)PEG1500, N,N′-bis(4-[(4-cyclohexylsulfanyl-3-sulfamoyl-benzoyl)amino]butanoyl);
Bis(3-aminopropyl)PEG1500, N,N′-bis(4-cyclohexylsulfanyl-3-sulfamoyl-benzoyl);
4arm-PEG5000-tetraacetamide, tetrakis-(N-[2-[2-(cyclooctylamino)-3,5,6-trifluoro-4-sulfamoyl-phenyl]sulfonylethyl]);
8arm-PEG10000-octaacetamide, octakis-(N-[2-[2-(cyclooctylamino)-3,5,6-trifluoro-4-sulfamoyl-phenyl]sulfonylethyl]).

15. A pharmaceutical composition comprising a compound according to claim 10 and pharmaceutically acceptable diluents, excipients or carriers.

16. A pharmaceutical composition according to claim 15 for use in the treatment of various cancers selected from the group of cervix carcinoma, esophageal carcinoma, pancreatic tumor, kidney carcinoma, endometrial adenocarcinoma, ovarian tumor, urinary bladder carcinoma, colonadenocarcinoma, lung tumor, liver carcinoma, and breast adenocarcinoma.

17. A pharmaceutical composition according to claim 15 for use in the treatment of other diseases, such as glaucoma, epilepsy, high altitude sickness, or Alzheimer's disease.

18. A compound according to claim 14 used for tumor visualization, fluorescent probes for visualization, or PET for tumor visualization.
